(19) United States
(12) Patent Application Publication
(10) Pub. No.: US 2023/0233247 A1
(43) Pub. Date: Jul. 27, 2023

(54) GRASP OVER-RIDE FOR IN-LINE VESSEL SEALER

(71) Applicant: Covidien LP, Mansfield, MA (US)

(72) Inventors: Ravi Sekhar Gutti, Hyderabad AP (IN); Rajanikanth Mandula, Hyderabad AP (IN)

(21) Appl. No.: 18/098,813

(22) Filed: Jan. 19, 2023

Related U.S. Application Data
(60) Provisional application No. 63/303,090, filed on Jan. 26, 2022.

(51) Int. Cl. A61B 18/14

(52) U.S. Cl. CPC A61B 18/1447; A61B 2018/0063

(57) ABSTRACT

A surgical instrument includes a housing having a shaft with an end effector assembly disposed at a distal end thereof that includes first and second jaw members configured to treat tissue upon electrical activation thereof. A first handle is movable relative to the housing and is configured to move the jaw members relative to one other to grasp tissue therebetween. A switch is disposed on the housing and in the actuation path of the first handle, the switch configured for activation by the first handle when the first handle is fully actuated relative to the housing. An over-ride is disposed in the housing and is movable between a first position configured to block full actuation of the first handle relative to the housing to prevent activation of the switch and a second position allowing full actuation of the first handle and activation of the switch.

### CROSS REFERENCE TO RELATED APPLICATION

This application claims the benefit of U.S. Provisional Application Ser. No. 63/303,090 filed Jan. 26, 2022, the entire contents of which being incorporated by reference herein.

### BACKGROUND

### Technical Field

The present disclosure relates generally to the field of surgical instruments. In particular, the disclosure relates to an in-line, endoscopic electrosurgical forceps that is economical to manufacture and is capable of sealing and cutting tissue structures.

### Background of Related Art

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

In-line electrosurgical forceps are one common type of electrosurgical instrument which offers the ease of electrically activating the forceps when fully and continuously compressing the same handle used to close the jaw members about tissue. In some instances, the surgeon may simply desire to grasp tissue and not electrically activate the jaw members. As such, it would be desirous to manufacturer an in-line electrosurgical forceps that facilitates both grasping tissue and in-line activation.

### SUMMARY

Provided in accordance with the present disclosure is a surgical instrument that includes a housing having a shaft with an end effector assembly disposed at a distal end thereof. The end effector includes first and second jaw members configured to treat tissue upon electrical activation thereof. A first handle is movable relative to the housing and is configured to move the jaw members relative to one other to grasp tissue therebetween. A switch is disposed on the housing and in the actuation path of the first handle, the switch configured for activation by the first handle when the first handle is fully actuated relative to the housing. An over-ride is disposed in the housing and is movable between a first position configured to block full actuation of the first handle relative to the housing to prevent activation of the switch and a second position allowing full actuation of the first handle and activation of the switch.

In aspects according to the present disclosure, the switch is operably coupled to a depressible button extending from the housing and configured to be selectively engaged by the first handle.

In aspects according to the present disclosure, the over-ride includes a push button mechanism having a plunger and cam, the plunger selectively actuatable relative to the cam between a first position wherein the cam operably engages a flange disposed on the first handle to prevent full actuation thereof and a second position wherein the cam allows full actuation of the first handle. In other aspects according to the present disclosure, the plunger includes a spring member that biases the cam in the first position upon actuation thereof. In yet other aspects according to the present disclosure, upon each actuation of the plunger, the cam rotates relative to the plunger between the first and second positions.

In aspects according to the present disclosure, the flange includes a groove defined therein configured to operably engage a distal end of the cam when the cam is disposed in the first position.

In aspects according to the present disclosure, the plunger and the cam include spring members that bias the cam in the first position upon actuation thereof.

In aspects according to the present disclosure, the surgical instrument further includes a second over-ride disposed on an opposing side of the housing, the second over-ride movable between a first position configured to block full actuation of the first handle relative to the housing to prevent activation of the switch and a second position allowing full actuation of the first handle and activation of the switch. In other aspects according to the present disclosure, the second over-ride includes a push button mechanism including a plunger and cam, the plunger of the second over-ride selectively actuatable relative to the cam of the second over-ride between a first position wherein the cam of the second over-ride operably engages the flange disposed on the first handle to prevent full actuation thereof and a second position wherein the cam of the second over-ride allows full actuation of the first handle.

In aspects according to the present disclosure, the plunger of the second over-ride includes a spring member that biases the cam of the second over-ride in the first position upon actuation thereof. In other aspects according to the present disclosure, upon each actuation of the plunger of the second over-ride, the cam of the second over-ride rotates relative to the plunger of the second over-ride between the first and second positions.

In aspects according to the present disclosure, the flange includes a groove defined therein configured to operably engage a distal end of the cam of the second over-ride when the cam of the second over-ride is disposed in the first position.

In aspects according to the present disclosure, the plunger of the second over-ride and the cam of the second over-ride include spring members that biases the cam of the second over-ride in the first position upon actuation thereof.

Provided in accordance with other aspects according to the present disclosure is an over-ride for an electrosurgical instrument that includes a push button mechanism disposed on a housing of the electrosurgical instrument, the push button mechanism including a plunger and cam. The plunger is selectively actuatable relative to the cam between a first position wherein the cam operably engages a flange disposed on a first handle of the electrosurgical instrument to prevent full actuation thereof and a second position wherein the cam allows full actuation of the first handle. A switch is disposed on the housing and in electrical communication with an energy source. The switch is disposed in the actuation path of the first handle and configured for activation by the first handle only when the over-ride is disposed in the second position allowing the first handle to fully actuate relative to the housing.

In aspects according to the present disclosure, the switch is operably coupled to a depressible button extending from the housing and configured to be selectively engaged by the first handle.

In aspects according to the present disclosure, the plunger includes a spring member that biases the cam in the first position upon actuation thereof.

In aspects according to the present disclosure, upon each actuation of the plunger, the cam rotates relative to the plunger between the first and second positions.

In aspects according to the present disclosure, the flange includes a groove defined therein configured to operably engage a distal end of the cam when the cam is disposed in the first position.

In aspects according to the present disclosure, the plunger and the cam include spring members that bias the cam in the first position upon actuation thereof.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a perspective view of an in-line, electrosurgical forceps according to an embodiment of the present disclosure including a housing, an elongated shaft, an end effector and an actuation handle;

FIG. 2A is an enlarged, perspective view of the end effector of FIG. 1 depicted with a pair of jaw members in an open configuration;

FIG. 2B is an enlarged, perspective view of the end effector of FIG. 1 depicted with the pair of jaw members in a closed configuration;

FIG. 3A is a perspective view of the end effector and elongated shaft of FIG. 1 with parts separated;

FIG. 3B is cross-sectional view taken along line 3B-3B of FIG. 3A showing a distal portion of the electrosurgical forceps of FIG. 1 depicting a tube guide;

FIG. 4 is a perspective view of a proximal portion of the instrument of FIG. 1 with a portion of the housing removed revealing internal components;

FIG. 5 is a partial, side view of a proximal portion of the instrument of FIG. 1;

FIG. 6A is an enlarged, partially transparent, perspective view of an over-ride configured to limit activation of the electrosurgical forceps according to the present disclosure;

FIG. 6B is an enlarged view of the internal components of the housing including the over-ride;

FIG. 6C is an enlarged, top cross section exposing the internal features of the over-ride;

FIG. 7A is a greatly-enlarged view of a plunger and cam of the over-ride disposed in an engaged position;

FIG. 7B is an enlarged, top cross section exposing further details of the internal features of the over-ride;

FIG. 7C is an enlarged, top cross section showing the internal features of the over-ride engaged to prevent electrical activation; and

FIG. 7D is an internal, perspective view of the actuation handle of the electrosurgical forceps fully actuated to deliver electrical energy.

### DETAILED DESCRIPTION

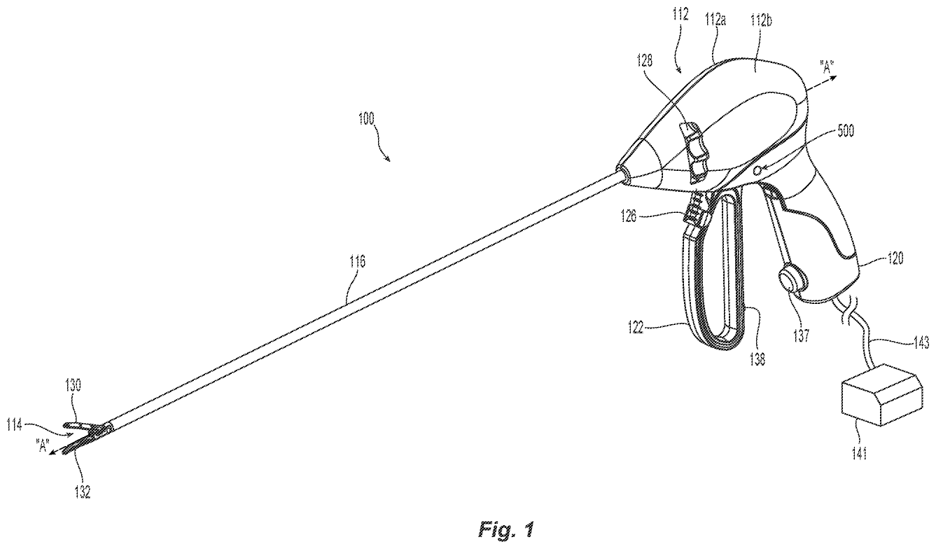

Referring initially to FIG. 1, an electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

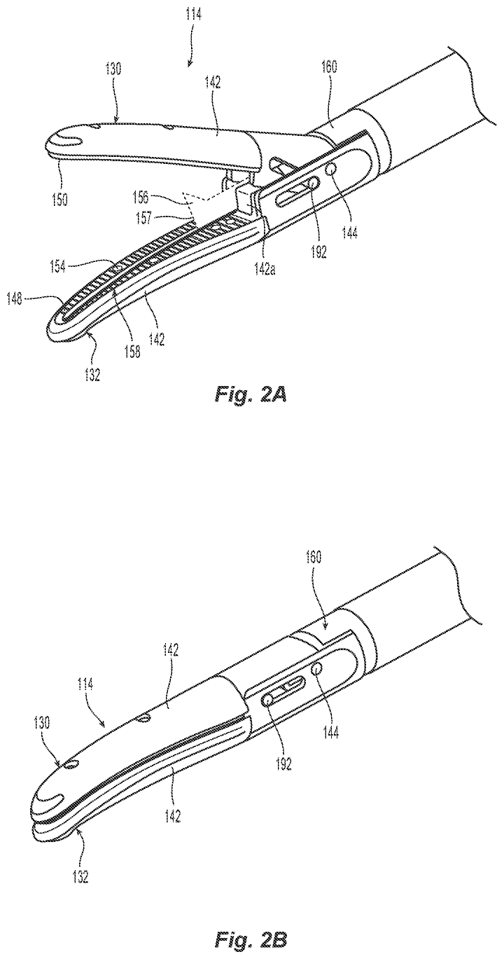

To mechanically control the end effector 114, the housing 112 supports a stationary handle 120, a movable handle 122, a trigger 126 and a rotation knob 128. The movable handle 122 is operable to move the end effector 114 between an open configuration (FIG. 2A) wherein a pair of opposed jaw members 130, 132 are disposed in spaced relation relative to one another, and a closed or clamping configuration (FIG. 2B) wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (see FIGS. 2A and 2B) through the end effector 114 when the end effector 114 is in the closed configuration. The rotation knob 128 serves to rotate the elongated shaft 116 and the end effector 114 about a longitudinal axis A-A extending through the forceps 114.

To electrically control the end effector 114, the stationary handle 120 supports a depressible button 137 thereon, which is operable by the user to initiate and terminate the delivery of electrosurgical energy to the end effector 114. The depressible button 137 is mechanically coupled to a switch (not shown) disposed within the stationary handle 120 and is engageable by a button activation post 138 extending from a proximal side of the moveable handle 122 upon proximal movement of the moveable handle 122 to an actuated or proximal position. The switch is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Sealing Generator and the ForceTriad® Generator sold by Covidien. The cable 143 may include a connector (not shown) thereon such that the forceps 100 may be selectively coupled electrically to the generator 141.

Referring now to FIGS. 2A-2B, the end effector 114 may be moved from the open configuration (FIG. 2A) wherein tissue (not shown) is received between the jaw members 130, 132, and the closed configuration (FIG. 2B), wherein the tissue is clamped and treated. The jaw members 130, 132 pivot about a pivot pin 144 to move the end effector 114 to the closed configuration of FIG. 2B wherein the sealing plates 148, 150 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 (FIG. 2A) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.006 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding. The stop members 154 may define any suitable number, arrangement, and/or configuration, depending on a particular purpose.

The upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (−) terminals associated with the generator 141. Thus, bipolar energy may be provided through the sealing plates 148 and 150 to tissue. Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert 140 and an insulator 142 that serves to electrically insulate the sealing plates 150, 148 from the jaw insert 140 of the jaw members 130, 132, respectively.

Electrosurgical energy may be delivered to the tissue through the electrically conductive seal plates 148, 150 to effect a tissue seal. Once a tissue seal is established, a knife blade 156 having a sharpened distal edge 157 may be advanced through a knife channel 158 defined in one or both jaw members 130, 132 to transect the sealed tissue. Although the knife blade 156 is depicted in FIG. 2A as extending from the elongated shaft 116 when the end effector 114 is in an open configuration, in some embodiments, extension of the knife blade 156 into the knife channel 158 when the end effector 114 is in the open configuration is typically prevented.

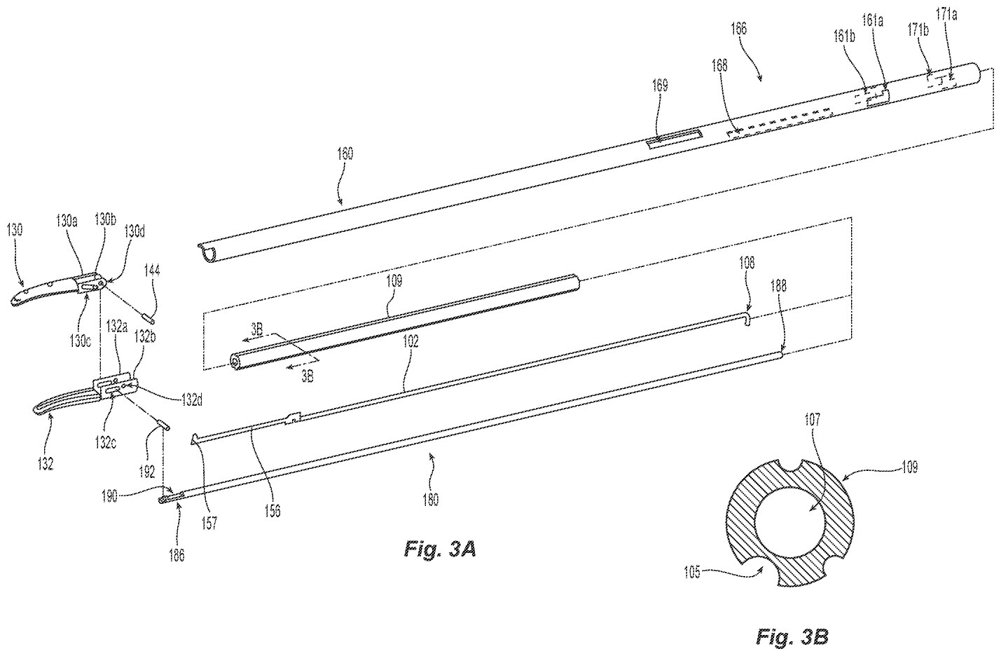

Referring to FIG. 3A, the elongated shaft 116 includes various longitudinal components that operatively couple the end effector 114 to the various actuators supported by the housing 112 (FIG. 1). An outer shaft member 160 defines an exterior surface of the elongated shaft 116 and houses other components therein as described below. The outer shaft member 160 is configured for longitudinal motion with respect to an inner actuation member 180 axially received within the outer shaft member 160. The inner actuation member 180 may be a rod, a shaft, a tube, folded metal, stamped metal, or other suitable structure. A proximal portion 166 of the outer shaft member 160 is configured for receipt within the housing 112 (FIG. 1), and includes features for operatively coupling the outer shaft member 160 to various elements of the housing 112. More specifically, the proximal portion 166 of the outer shaft member 160 includes, in order from distal to proximal, a longitudinal slot 169 to couple the outer shaft member 160 to the rotation knob 128, a longitudinal knife slot 168 defined therethrough, a pair of opposing distal locking slots 161a, 161b, and a pair of opposing proximal locking slots 171a, 171b.

A distal portion 186 of the inner actuation member 180 includes a longitudinal recess 190 defined therein that provides clearance for the pivot pin 144 and thus, permits longitudinal reciprocation of the pivot pin 144 (via longitudinal reciprocation of the outer shaft member 160) independent of the inner actuation member 180. Distally of the longitudinal recess 190, a cam pin 192 is mechanically coupled (e.g., via welding, friction-fit, laser welding, etc) to the distal portion 186 of the inner actuation member 180.

The pivot pin 144 extends through a proximal portion of each of the jaw members 130, 132 to pivotally support the jaw members 130, 132 at the distal end of the inner actuation member 180. A proximal portion of each of the jaw members 130, 132 includes two laterally spaced parallel flanges or “flags” 130a, 130b and 132a, 132b respectively, extending proximally from a distal portion of the jaw members 130 and 132 (FIG. 3A). A lateral cam slot 130c and a lateral pivot bore 130d extend through each of the flags 130a, 130b of the upper jaw member 130 (FIG. 3A). Similarly, a lateral cam slot 132c and a lateral pivot bore 132d extend through each of the flags 132a, 132b of the lower jaw member 132. The pivot bores 130d, 132d receive the pivot pin 144 in a slip-fit relation that permits the jaw members 130, 132 to pivot about the pivot pin 144 to move the end effector 114 between the open and closed configurations (FIGS. 2A and 2B, respectively).

A tube guide 109 is disposed within the outer shaft member 160 and includes a lumen 107 axially disposed therethrough. The inner actuation member 180 is received within the guide lumen 107, which serves to orient and align the inner actuation member 180 within the outer shaft member 160. The knife rod 102 is received within a longitudinal guide recess 105 formed in the outer surface of the guide tube 109. The guide recess 105 serves to guide longitudinal motion of the knife rod 102 within the outer shaft member 160 and to radially space the knife rod 102 from the inner actuation member 180 to prevent the inner actuation member 180 from interfering with reciprocal motion of the knife rod 102.

Rotation knob 128 imparts rotational motion to each of the components of the elongated shaft 116, and to the end effector 114, which is coupled thereto. The rotation knob 128 is supported in the housing 112 and, as shown in FIG. 1, extends radially outward from opposing sides of the housing 112 (only shown extending radially outward from housing half 112b).

End effector 114 is coupled to the distal end of the inner actuation member 180 by the cam pin 192. The cam pin 192 represents a longitudinally stationary reference for longitudinal movement of the outer shaft member 160 and the knife rod 102. The cam pin 192 extends through the flags 132a, 132b of the lower jaw member 132 and the flags 130a and 130b of the upper jaw member 130.

The outer shaft member 160 may be drawn proximally relative to the inner actuation member 180 and the cam pin 192 to move the end effector 114 to the closed configuration (see FIG. 2B). Since the longitudinal position of the cam pin 192 is fixed, and since the cam slot 130c is obliquely arranged with respect to the longitudinal axis A-A, proximal retraction of the outer shaft member 160 induces distal translation of the cam pin 192 through the cam slots 130c, 132c such that the jaw member 130 pivots toward jaw member 132 about the pivot pin 144. Conversely, when the end effector 114 is in the closed configuration, longitudinal translation of the outer shaft member 160 in a distal direction induces proximal translation of the cam pin 192 through the cam slots 130c, 132c such that jaw member 130 pivots away from jaw member 132 toward the open configuration.

The lower jaw member 132 is constructed of three major components: the jaw insert (not shown), the insulator 142, and the sealing plate 148. The flags 132a, 132b of the jaw member 132 define a proximal portion of the jaw insert and a generally u-shaped profile of the jaw insert extends distally to support the tissue engaging portion of the jaw member 132. Upper jaw member 130 includes the same three major components as lower jaw member 132, including sealing plate 150, jaw insert (not shown), and insulator 142, and is constructed in the same manner as lower jaw member 132. However, lower jaw member 132 is fixedly engaged, e.g., welded, to outer shaft member 160, while upper jaw member 130 is pivotable relative to lower jaw member 132 and outer shaft member 160 between the open and closed configurations.

In order to facilitate alignment of lower jaw member 132 with outer shaft member 160 during welding (or other suitable fixed engagement), the jaw insert and outer shaft member 160 may include complementary alignment features, e.g., a complementary recess (not explicitly shown) defined within jaw insert and a complementary protrusion (not explicitly shown) extending from outer shaft member 160. As an alternative to the unilateral configuration detailed above, both of the upper and lower jaw members 130, 132, respectively, may be pivotable relative to one another and outer shaft member 160, thus defining a bilateral configuration.

The insulator 142 of jaw members 130, 132 may be constructed of an electrically insulative plastic such as a polyphthalamide (PPA) (e.g., Amodel®), polycarbonate (PC), acrylonitrile butadiene styrene (ABS), a blend of PC and ABS, nylon, ceramic, etc. The insulator 142 may be overmolded onto the jaw insert in either a single-shot or a two-shot injection molding process such that each of the sealing plates 148, 150 are coupled to and in spaced relation with their respective jaw inserts 140.

In some embodiments, the insulator 142 on the lower jaw member 132 forms a tissue stop 142a extending therefrom adjacent to the knife channel 158 and proximal to the sealing plate 148. The tissue stop 142a serves to prevent tissue from entering the distal end of the outer shaft member 160 and to prevent splay of the flags 130a, 130b of the upper jaw member 130. In some embodiments, the tissue stop 142a may be formed by the insulator 142 on the upper jaw member 130 or on both the upper jaw member 130 and the lower jaw member 132. The tissue stop 142a may also serve to align the knife blade 156 as the knife blade 156 enters the knife channel 158 defined in the jaw members 130, 132. To this end, the surface of the tissue stop 142a extending along the path of the knife blade 156 may define a chamfered configuration to further facilitate alignment of the knife blade 156 as the knife blade 156 enters the knife channel 158.

The movable handle 122 may be manipulated to impart longitudinal motion to the outer shaft member 160, and the knife trigger 126 may be manipulated to impart longitudinal motion to the knife rod 102. As discussed above, longitudinal motion of the outer shaft member 160 serves to move the end effector 114 between the open configuration of FIG. 2A and the closed configuration of FIG. 2B, and longitudinal motion of the knife rod 102 serves to move knife blade 156 through knife channel 158 (FIG. 2A).

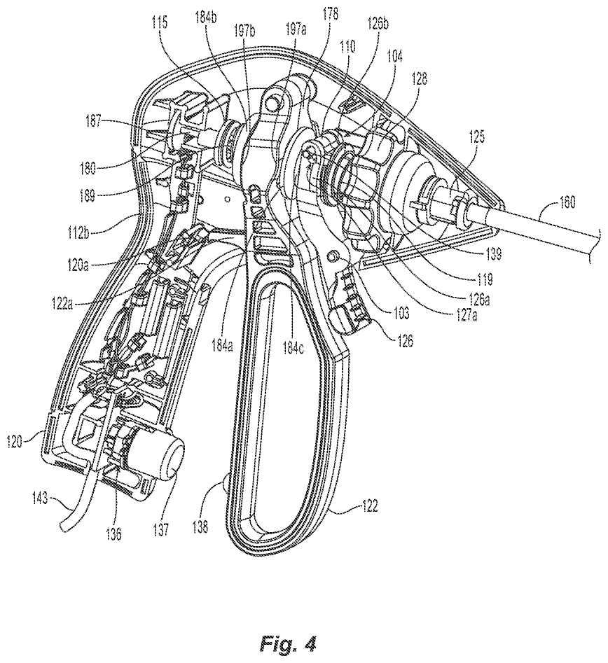

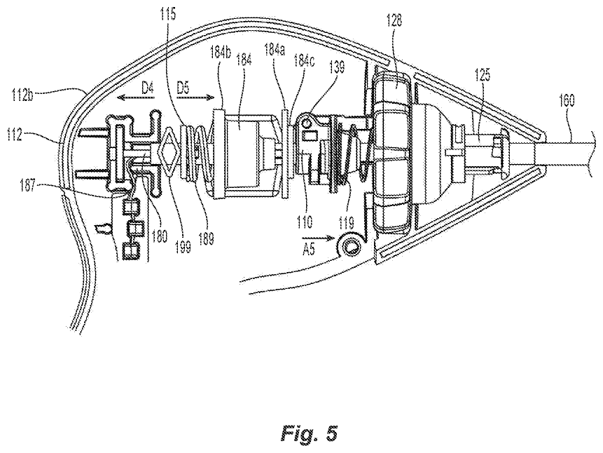

The movable handle 122 is operatively coupled to the outer shaft member 160 by a clevis 178 defined at an upper end of the movable handle 122 (FIG. 4). The clevis 178 is pivotally supported on the housing 112. The clevis 178 extends upwardly about opposing sides of a drive collar 184 (FIG. 5) supported on the outer shaft member 160 and includes rounded drive surfaces 197a and 197b thereon. Drive surface 197a engages a proximal-facing surface of a distal spring washer 184a and drive surface 197b engages a distal facing surface of a proximal rim 184b of the drive collar 184 (FIG. 5). The distal spring washer 184a engages a proximal facing surface of a distal spring stop 184c that, in turn, engages the opposing distal locking slots 161a, 161b (FIG. 3A) extending through the proximal portion 166 (FIG. 3A) of the outer shaft member 160 to couple the distal spring stop 184c to the outer shaft member 160. The drive surfaces 197a, 197b are arranged along the longitudinal axis A-A such that pivotal motion of the movable handle 122 induces corresponding longitudinal motion of the drive collar 184 (FIG. 5) along the longitudinal axis A-A.

Proximal longitudinal motion may be imparted to the outer shaft member 160 by pushing the proximal rim 184b of the drive collar 184 proximally with the movable handle 122 (FIG. 4) as indicated by arrow D4 (FIG. 5). A spring 189 is constrained between a proximal facing surface of the drive collar 184 and a proximal spring stop 115. The proximal spring stop 115 engages the opposing proximal locking slots 171a, 171b (FIG. 3A) extending through the proximal portion 166 (FIG. 3A) of the outer shaft member 160 to couple the proximal spring stop 115 to the outer shaft member 160. Thus, the proximal spring stop 115 serves as a proximal stop against which spring 189 compresses.

Distal longitudinal motion is imparted to the outer shaft member 160 by driving the drive collar 184 distally with the movable handle 122 (FIG. 4). Distal longitudinal motion of the drive collar 184 induces a corresponding distal motion of the outer shaft member 160 by virtue of the coupling of the drive collar 184 to opposing distal locking slots 181a, 181b extending through the proximal portion 166 of the outer shaft member 160 (FIG. 3A).

Proximal longitudinal motion of the outer shaft member 160 draws jaw member 132 proximally such that the cam pin 192 advances distally to pivot jaw member 130 toward jaw member 132 to move the end effector 114 to the closed configuration. Once the jaw members 130 and 132 are closed, the outer shaft member 160 essentially bottoms out (i.e., further proximal movement of the outer shaft member 160 is prohibited since the jaw members 130, 132 contact one another). Further proximal movement of the movable handle 122 (FIG. 4), however, will continue to move the drive collar 184 proximally. This continued proximal movement of the drive collar 184 further compresses the spring 189 to impart additional force to the outer shaft member 160, which results in additional closure force applied to tissue grasped between the jaw members 130, 132 (see FIG. 2B).

Referring again to FIG. 4, the trigger 126 is pivotally supported in the housing 112 about a pivot boss 103 protruding from the trigger 126. The trigger 126 is operatively coupled to the knife rod 102 by a knife connection mechanism 104 such that pivotal motion of the trigger 126 induces longitudinal motion of the knife rod 102. The knife connection mechanism 104 includes upper flanges 126a, 126b of the trigger 126 and a knife collar 110.

As the moveable handle 122 is moved to a fully actuated or proximal position of, the button activation post 138 depresses the depressible button 137, thereby activating the switch disposed within the stationary handle 120 to initiate the delivery of electrosurgical energy to the end effector 114 to generate a tissue seal.

As the movable handle 122 is moved from an intermediate position to a fully actuated or proximal position of, the pressure applied by the jaw members 130, 132 is increased. As the movable handle 122 pivots further the spring 189 is compressed against the proximal spring stop 115, and a tensile force is transmitted through the outer shaft member 160 to the jaw members 130, 132. The tensile force supplied by the spring 189 ensures that the jaw members 130, 132 apply an appropriate pressure to effect a tissue seal.

When the movable handle 122 is in the fully actuated or proximal position, the knife trigger 126 may be selectively moved to a proximal position to advance the knife blade 156 distally through knife channel 158.

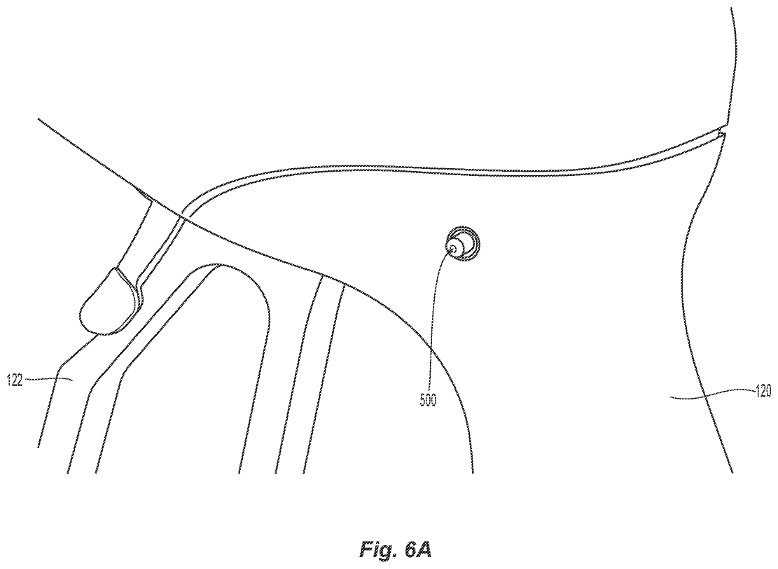

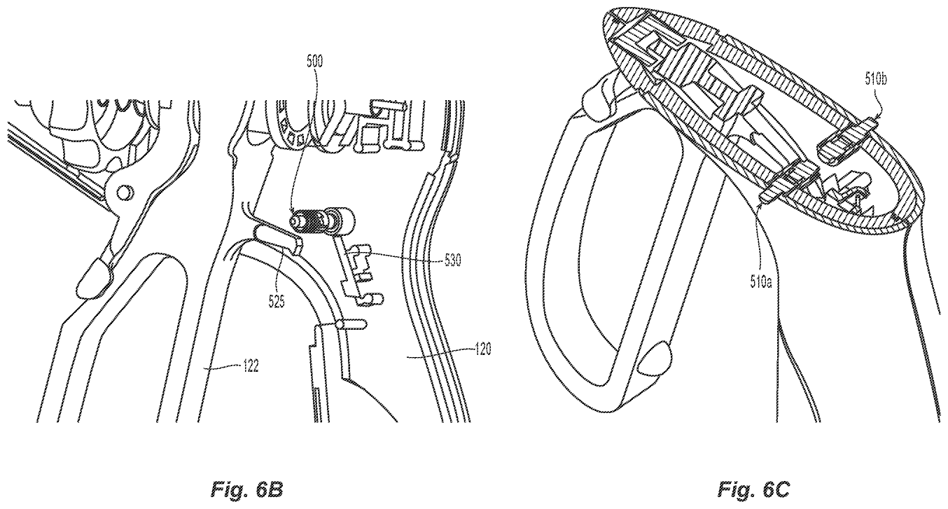

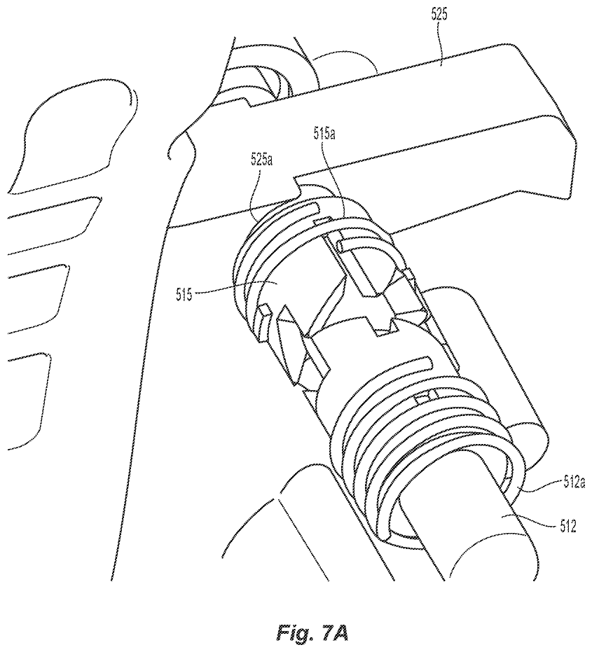

FIGS. 6A-6C show one embodiment of a grasp over-ride feature 500 for the electrosurgical forceps 100 according to the present disclosure enabling a surgeon to selectively choose to either grasp tissue or grasp and seal tissue upon actuation of handle 122. More particularly, grasp over-ride (hereinafter “over-ride 500”) is disposed on handle 120 of the forceps 100 in a convenient location for easy access thereto with a surgeon's thumb. As shown, over-ride 500 is disposed on both sides of handle 120 to facilitate access for either right or left-handed surgeons.

Over-ride 500 includes opposing push button members 510a, 510b that are each configured to rotate and inwardly project upon compression thereof to mechanically engage a corresponding flange 525 disposed on the proximal side of handle 122. Pushing the members 510a, 510b again retracts and disengages the push button members 510a, 510b from the flange 525.

Push button member 510a includes a plunger 512 mechanically engaged atop a cam 515 in biased engagement therewith. A plunger spring 512a biases the plunger 512 relative to the cam 515. A cam spring 515b biases the cam 515 within a pocket 530 defined within handle 120. Similarly, push button member 510b includes a plunger 520 mechanically engaged atop a cam 522 in biased engagement therewith. A plunger spring 520a biases the plunger 520 relative to the cam 522. A cam spring 522a biases the cam 522 within a pocket 532 defined within handle 120. For the purposes herein, only push button member 510a is further described.

Push button 510a is normally unbiased within pocket 530 allowing a surgeon to freely actuate the handle 122 relative to handle 120 in an in-line fashion to grasp and activate the forceps 100 through continued actuation of the handle 122 as explained in detail above. If a surgeon desires to utilize the forceps 100 without electrical activation (e.g., without sealing tissue through the stroke of handle 122), the surgeon actuates the over-ride 500 by pressing one of the two push button members 510a, 510b disposed on either side of handle 120.

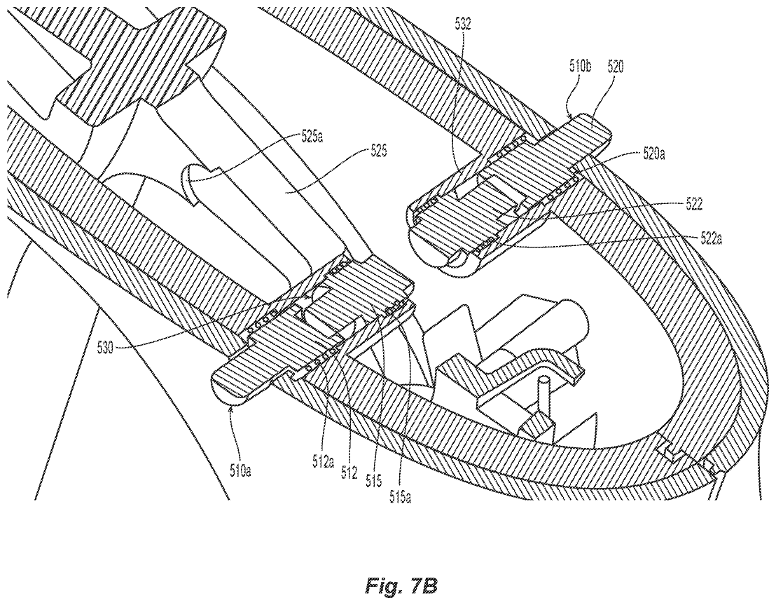

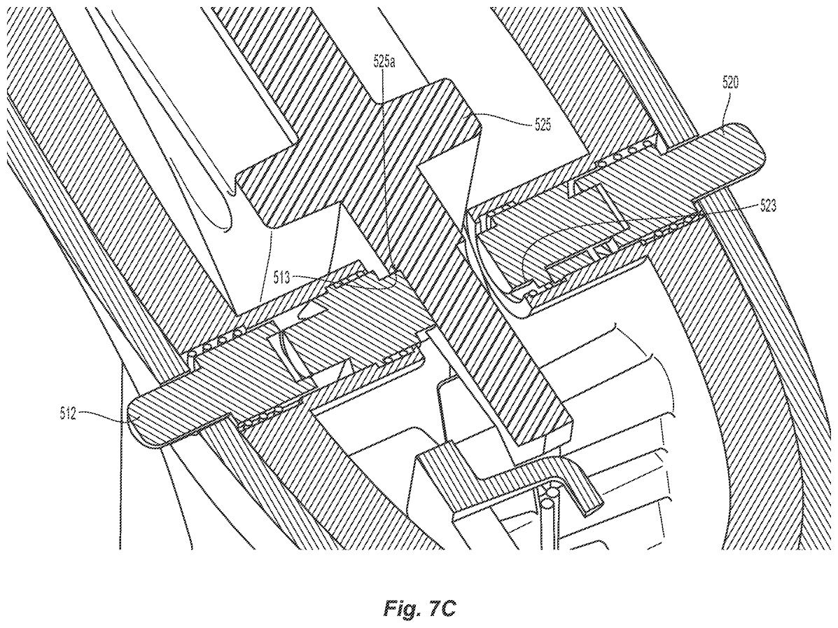

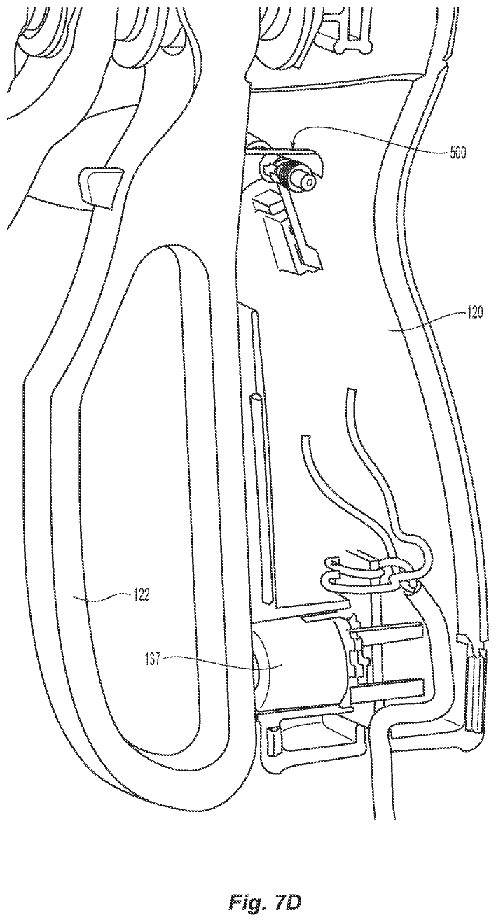

Pressing the plunger 512 relative to the handle 120 (e.g., into the handle 120), rotates the cam 515 to extend from pocket 530 and into the path of flange 525 thereby preventing the flange 525 and handle 122 from fully compressing and activating switch 137. A groove 525a is defined within the flange 525 and is configured to receive a distal end of the cam 515 when extended (FIGS. 7A-7D) thereby preventing further proximal movement of the flange 525. The rotation of the cam 515 relative to the plunger 512 extends the cam 515 into the path of the flange 525 which is held in a biased configuration under the force of the springs 512a, 515a against the pocket 530. Pressing the plunger 512 again, rotates the cam 515 (again) relative to the plunger 512 and retracts the cam 515 back into the pocket 530 allowing the flange 525 and handle 122 to fully actuate.

As can be appreciated, the simple, yet effective design of the over-ride 500 provides easy adaptability for current in-line models, is very cost effective and provides additional flexibility for surgeons during use.

Other types of selectively lockable and un-lockable over-ride mechanisms for in-line surgical instrument are also contemplated which are configured to impede the path of the handle 122 and prevent activation of the forceps 100.

A return spring and/or tactile/audible indicator 530 (FIG. 6B) may be disposed in the actuation path of the handle 122. The indicator 530 may be a leaf spring which may be configured to provide multiple functions, e.g., act as a spring release to disengage the switch 137 and stop electrical activation, act as a tactile/audible indicator to provide feedback to the user regarding electrical activation.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

What is claimed is:

1. A surgical instrument, comprising:
a housing including an elongated shaft having an end effector assembly disposed at a distal end thereof, the end effector including first and second jaw members configured to treat tissue upon electrical activation thereof;
a first handle movable relative to the housing and configured to move at least one of the first or second jaw members relative to the other of the first or second jaw members to grasp tissue therebetween;
a switch disposed on the housing and in the actuation path of the first handle, the switch configured for activation by the first handle when the first handle is fully actuated relative to the housing; and
an over-ride disposed in the housing and movable between a first position configured to block full actuation of the first handle relative to the housing to prevent activation of the switch and a second position allowing full actuation of the first handle and activation of the switch.

2. The surgical instrument according to claim 1, wherein the switch is operably coupled to a depressible button extending from the housing and configured to be selectively engaged by the first handle.

3. The surgical instrument according to claim 1, wherein the over-ride includes a push button mechanism including a plunger and cam, the plunger selectively actuatable relative to the cam between a first position wherein the cam operably engages a flange disposed on the first handle to prevent full actuation thereof and a second position wherein the cam allows full actuation of the first handle.

4. The surgical instrument according to claim 3, wherein the plunger includes a spring member that biases the cam in the first position upon actuation thereof.

5. The surgical instrument according to claim 4, wherein upon each actuation of the plunger, the cam rotates relative to the plunger between the first and second positions.

6. The surgical instrument according to claim 3, wherein the flange includes a groove defined therein configured to operably engage a distal end of the cam when the cam is disposed in the first position.

7. The surgical instrument according to claim 3, wherein the plunger and the cam include spring members that bias the cam in the first position upon actuation thereof.

8. The surgical instrument according to claim 1, further comprising a second over-ride disposed on an opposing side of the housing, the second over-ride movable between a first position configured to block full actuation of the first handle relative to the housing to prevent activation of the switch and a second position allowing full actuation of the first handle and activation of the switch.

9. The surgical instrument according to claim 8, wherein the second over-ride includes a push button mechanism including a plunger and cam, the plunger of the second over-ride selectively actuatable relative to the cam of the second over-ride between a first position wherein the cam of the second over-ride operably engages the flange disposed on the first handle to prevent full actuation thereof and a second position wherein the cam of the second over-ride allows full actuation of the first handle.

10. The surgical instrument according to claim 9, wherein the plunger of the second over-ride includes a spring member that biases the cam of the second over-ride in the first position upon actuation thereof.

11. The surgical instrument according to claim 10, wherein upon each actuation of the plunger of the second over-ride, the cam of the second over-ride rotates relative to the plunger of the second over-ride between the first and second positions.

12. The surgical instrument according to claim 9, wherein the flange includes a groove defined therein configured to operably engage a distal end of the cam of the second over-ride when the cam of the second over-ride is disposed in the first position.

13. The surgical instrument according to claim 9, wherein the plunger of the second over-ride and the cam of the second over-ride include spring members that biases the cam of the second over-ride in the first position upon actuation thereof.

14. An over-ride for an electrosurgical instrument, comprising:
a push button mechanism disposed on a housing of the electrosurgical instrument, the push button mechanism including a plunger and cam, the plunger selectively actuatable relative to the cam between a first position wherein the cam operably engages a flange disposed on a first handle of the electrosurgical instrument to prevent full actuation thereof and a second position wherein the cam allows full actuation of the first handle; and
a switch disposed on the housing and disposed in electrical communication with an energy source, the switch disposed in the actuation path of the first handle and configured for activation by the first handle only when the over-ride is disposed in the second position allowing the first handle to fully actuate relative to the housing.

15. The surgical instrument according to claim 14, wherein the switch is operably coupled to a depressible button extending from the housing and configured to be selectively engaged by the first handle.

16. The surgical instrument according to claim 14, wherein the plunger includes a spring member that biases the cam in the first position upon actuation thereof.

17. The surgical instrument according to claim 14, wherein upon each actuation of the plunger, the cam rotates relative to the plunger between the first and second positions.

18. The surgical instrument according to claim 14, wherein the flange includes a groove defined therein configured to operably engage a distal end of the cam when the cam is disposed in the first position.

19. The surgical instrument according to claim 14, wherein the plunger and the cam include spring members that bias the cam in the first position upon actuation thereof.
